(19) United States
(12) Patent Application Publication
(10) Pub. No.: US 2024/0238042 A1
(43) Pub. Date: Jul. 18, 2024

(54) TUMOR DENATURIZATION CONTROL IN CURATIVE CANCER TREATMENT

(71) Applicant: CURATIVE CANCER TREATMENT BY HEAT (CCTBH) AB, Saltsjöbaden (SE)

(72) Inventor: Hans WIKSELL, Täby (SE)

(73) Assignee: CURATIVE CANCER TREATMENT BY HEAT (CCTBH) AB, Saltsjöbaden (SE)

(21) Appl. No.: 18/289,510

(22) PCT Filed: May 6, 2022
(86) PCT No.: PCT/EP2022/062316

§ 371 Date: Nov. 3, 2023

(30) Foreign Application Priority Data
May 6, 2021 (SE) 2150579-7

(51) Int. Cl. A61B 18/16; A61B 18/00; A61B 18/12

(52) U.S. Cl. CPC A61B 18/16; A61B 18/1206; A61B 2018/00023; A61B 2018/00077; A61B 2018/00083; A61B 2018/00184; A61B 2018/00577; A61B 2018/00648; A61B 2018/00672; A61B 2018/00708; A61B 2018/00714; A61B 2018/1253; A61B 2018/126; A61B 2018/167

(57) ABSTRACT

A system for curative RFA treatment of a treatment volume including at least a part of a cancer tumor by applying RF energy between an electrode arranged on an uninsulated ablating portion of an RF probe and another electrode is provided. The system includes: a control arrangement; an RF probe, including an uninsulated ablating portion; a temperature measuring arrangement, arranged to measure an RF probe temperature and provide estimations of the treatment volume temperature to the control arrangement; a cooling arrangement, controlled by the control arrangement to cool the RF probe; and an RF generating arrangement, controlled by the control arrangement to, based on the estimated treatment volume temperature, supply the amount of RF energy to the RF probe that is needed for a desired treatment volume temperature to be maintained. The control arrangement monitors the RF energy supplied by the RF generating arrangement to the RF probe, and turns off the RF generating arrangement when there is no longer any substantial lowering of the output power.

### TECHNICAL FIELD

The present disclosure relates generally to systems and methods for minimally-invasive curative treatment of cancer tumors.

### BACKGROUND

Cancer tumors are most commonly treated with surgery, radiotherapy and/or chemotherapy, but other means of treatment have also been proposed. Hyperthermia is a type of cancer treatment in which tumors are exposed to temperatures around 43° C. Research described e.g. in the article “Heating the patient: a promising approach?”, Annals of Oncology 2002, 13(8): 1173-1184, by van der Zee has shown that temperatures around 43° C. can damage and kill cancer cells with minimal injury to normal tissues.

Radio frequency ablation (RFA) is a medical procedure in which tissue is ablated using the heat generated from medium frequency alternating current. Monopolar RFA may be used to treat tumors by inserting an RF probe directly into the tumor to be treated and applying an indifferent electrode to an outer surface of the body, e.g. in the form of a large metal plate or foil on which the patient lies. When radio frequency (RF) energy is applied between the indifferent electrode and the RF probe (acting as a counter electrode), a current path is established between the electrodes. The current density at the indifferent electrode will be much smaller than at the RF probe, and thus there will be virtually no heating of the body by the indifferent electrode, as long as there is good contact between the body and the indifferent electrode. In bipolar RFA, the RF energy is instead applied between an electrode located on an RF probe and another electrode which is also located on an RF probe, either the same RF probe or another RF probe.

The heating of the tissue surrounding the RF probe by the RF energy is caused by the electric resistance of the tissue. The output power of the RF source regulates the temperature that is generated in the tissue. It is the heat that destroys the tumor cells. The denaturized tumor cells may be left to be later resorbed by the body. RFA is mainly used for palliative treatments.

EP1639956 describes a monopolar RFA arrangement for tumor treatment, comprising an RF probe that is intended to be inserted into the tumor, an indifferent electrode that is intended to be applied to the outer surface of the body, and an RF generating device that is intended to apply RF energy between the RF probe and the indifferent electrode such that heat is generated in tissue surrounding the RF probe, wherein said heat is in the thermotherapy temperature range (70-95° C.).

### PROBLEMS WITH THE PRIOR ART

RFA is presently mainly used for palliative treatments. One of the reasons for this is that it is difficult to determine when the tumor has been destroyed in its entirety. This is not necessary for palliative treatments.

There is thus a need for an improved system for curative treatment of cancer tumors.

### SUMMARY

The above described problem is addressed by the claimed system for curative RFA treatment of a treatment volume comprising at least a part of a cancer tumor by applying RF energy between an electrode arranged on an uninsulated ablating portion of an RF probe and another electrode. The system may comprise: a control arrangement; a temperature measuring arrangement, arranged to measure an RF probe temperature and based on this provide estimations of the treatment volume temperature to the control arrangement; a cooling arrangement, arranged to be controlled by the control arrangement to cool the RF probe; and an RF generating arrangement, controlled by the control arrangement to, based on the estimated treatment volume temperature, supply the amount of RF energy to the RF probe that is needed for a desired treatment volume temperature to be maintained. The RF energy supplied by the RF generating arrangement to the RF probe may be monitored, and the RF generating arrangement may be arranged to be turned off at the point in time when there is no longer any substantial lowering of the output power from the RF generating arrangement.

The above described problem is further addressed by the claimed method, in a system for curative RFA treatment of a treatment volume comprising at least a part of a cancer tumor by applying RF energy between an electrode arranged on an uninsulated ablating portion of an RF probe and another electrode, for determining the point in time when the blood vessels within the treatment volume have coagulated. The method may comprise: measuring an RF probe temperature using a temperature measuring arrangement, and based on this estimating the treatment volume temperature; cooling the RF probe, using a cooling arrangement; supplying, based on the estimated treatment volume temperature, the amount of RF energy to the RF probe that is needed for a desired treatment volume temperature to be maintained, using an RF generating arrangement; monitoring the RF energy supplied by the RF generating arrangement to the RF probe; and determining the point in time when there is no longer any substantial lowering of the output power from the RF generating arrangement, in order to end the RFA treatment by turning off the RF generating arrangement after this point in time.

This is a precise way of determining that the tumor has been denaturized, and that the curative treatment is thus finished.

In embodiments, the determining of the point in time when there is no longer any substantial lowering of the output power from the RF generating arrangement involves determining when the derivative of the output power from the RF generating arrangement comes within a predetermined threshold, close to zero. Since the derivative of the output power from the RF generating arrangement will be zero also early during the treatment process, at the high point of the curve, any automatic system for determining when there is no longer any substantial lowering of the output power from the RF generating arrangement based on the derivative of the output power curve needs to differentiate between these points. This may be done e.g. based on a certain treatment time having passed, or based on the derivative first being negative.

The control arrangement may be arranged to, at selected intervals, shut off the RF generating arrangement and the cooling arrangement for a selected shut-off period, to thereby allow the treatment volume temperature T to equalize, so that it can be more accurately estimated by the temperature measuring arrangement. This enables a precise control of the treatment volume temperature.

In embodiments, the treatment volume temperature is estimated based on the shape of the measured RF probe temperature curve, e.g. based on a determination of the derivative of the measured RF probe temperature curve up to the end of the shut-off period. This means that it is not necessary to wait for the RF probe temperature to fully stabilize, and thus the shut-off period can be substantially shortened.

In embodiments, the cooling arrangement is arranged to cool the RF probe by circulating cooling liquid, and to be turned off by stopping the circulation of the cooling liquid. This is a simple way of controlling the cooling of the RF probe.

In embodiments, the probe measuring arrangement comprises a thermocouple comprising a conductor that is arranged inside the RF probe and connected to the uninsulated ablating portion of the RF probe. This is a simple temperature measuring arrangement.

In embodiments, the cooling arrangement comprises a cooling liquid supply channel arranged inside the RF probe to supply cooling liquid to the uninsulated ablating portion of the RF probe. This ensures that the cooling liquid is supplied to the whole of the uninsulated ablating portion of the RF probe.

In embodiments, a striking arrangement is arranged to push the RF probe into the tumor in distinct strokes. The striking arrangement may be arranged to comprise a striking device, arranged within an impact housing to be shot towards an impact peg that extends from a connecting end of the RF probe, so that the impact between the striking device and the impact peg pushes the RF probe forward, with a very high acceleration, a distance that is defined by the length that the impact peg extends into the impact housing. The striking arrangement may further be arranged to comprise a spring device, with which the impact peg interacts, wherein the spring device pushes the RF probe away from the striking arrangement, so that the length that the impact peg extends into the impact housing becomes dependent of the force applied to the RF probe to counteract the spring force in the spring device. This is a simple yet efficient way of arranging a striking arrangement.

In embodiments, the RF probe is arranged to be comprised in a probe arrangement also comprising an insertion tube and a biopsy needle. In embodiments, the insertion tube is arranged to be inserted into a tumor together with the biopsy needle; remain inserted in the tumor when the biopsy needle is retracted; and allow the insertion of the RF probe into the cavity in the tumor created by the biopsy needle. This enables the taking of a tissue sample in the exact location that is treated with the curative RFA treatment.

In embodiments, the RF generating arrangement comprises an oscillator, a balanced modulator and an RF amplifier.

In embodiments, an isolation transformer is arranged between the RF generating arrangement and the RF probe.

The RF probe temperature is preferably measured by a temperature sensing arrangement comprised in the temperature measuring arrangement. The estimation of the treatment volume temperature based on the measured RF probe temperature may be made by processing means comprised in the temperature measuring arrangement, or by processing means comprised in the control arrangement.

The RFA treatment may be either a monopolar RFA treatment or a bipolar RFA treatment. If the RFA treatment is a monopolar RFA treatment, the other electrode is preferably in the form of an indifferent electrode. If the RFA treatment is a monopolar RFA treatment, the other electrode may be arranged on a separate RF probe, or on an uninsulated ablating portion of the same RF probe. The RFA treatment may also be a combination of a monopolar RFA treatment and a bipolar RFA treatment.

The RFA treatment may treat tumors in humans as well as animals.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 schematically illustrates a system for curative treatment of a treatment volume comprising at least a part of a cancer tumor, in accordance with one or more embodiments described herein.

FIG. 2a shows cell survival curves at various temperatures.

FIG. 2b schematically illustrates an RF probe inserted into a treatment volume comprising a cancer tumor, in accordance with one or more embodiments described herein.

FIGS. 3a-b illustrate different aspects of an RF probe for use in curative treatment of a treatment volume comprising at least a part of a cancer tumor, in accordance with one or more embodiments described herein.

FIGS. 4a-c illustrate a probe arrangement for use in curative treatment of a treatment volume comprising at least a part of a cancer tumor, in accordance with one or more embodiments described herein.

FIG. 5a schematically illustrates how the output power of an RF generating arrangement may vary during the course of an RFA treatment.

FIG. 5b schematically illustrates how a measured RF probe temperature is typically affected by an RF generating arrangement and a cooling arrangement being turned off.

FIG. 6 schematically illustrates a method, in a system for curative treatment of a treatment volume comprising at least a part of a cancer tumor, in accordance with one or more embodiments described herein.

FIG. 7 schematically illustrates a method for curative treatment of a treatment volume comprising at least a part of a cancer tumor, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

RFA is presently mainly used for palliative treatments. One of the reasons for this is that it is difficult to determine when the tumor has been destroyed in its entirety. This is not necessary for palliative treatments.

Another reason is that it is difficult to control the temperature in the tumor during an RFA treatment. It is important that it can be determined that the temperature within the entire treatment volume is high enough to kill the cancer cells, without becoming so high that there is a risk that e.g. coagulated tissue explodes.

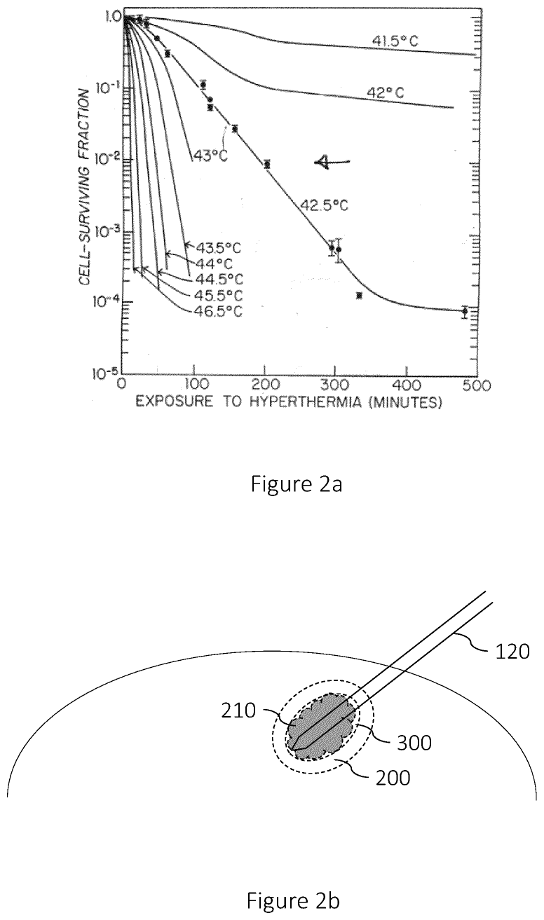

The temperature needed to kill cancer cells depends on the treatment time. FIG. 2a shows cell survival curves for mammalian cells heated at different temperatures for varying lengths of time (redrawn from Dewey W C, Hopwood L E, Sapareto S A, Gerweck L E: Radiology 123:463-474, 1977). Based on these curves, hyperthermia treatments, in which tumors are exposed to temperatures of around 43° C., have been proposed. However, as seen in FIG. 2a, very long treatment times are needed at such temperatures. Also, the hyperthermia technique is highly sensitive to small temperature changes, and thus requires an extremely sensitive temperature control.

The claimed invention relates to curative RFA treatment in the thermotherapy temperature range (60-90° C.). It can be seen in FIG. 2a that as the temperature is raised to 46.5° C., the treatment time is substantially shortened. It is thus clear from FIG. 2a that if the temperature is raised even further, this will shorten the treatment time even further. The temperature range 60-90° C. is selected to ensure that the temperature within the whole treatment volume is at least e.g. 50° C., even though the blood vessels within the tumor constantly actively transport heat away from the tumor.

Embodiments of the disclosed solution are presented in more detail in connection with the figures.

### System Embodiments

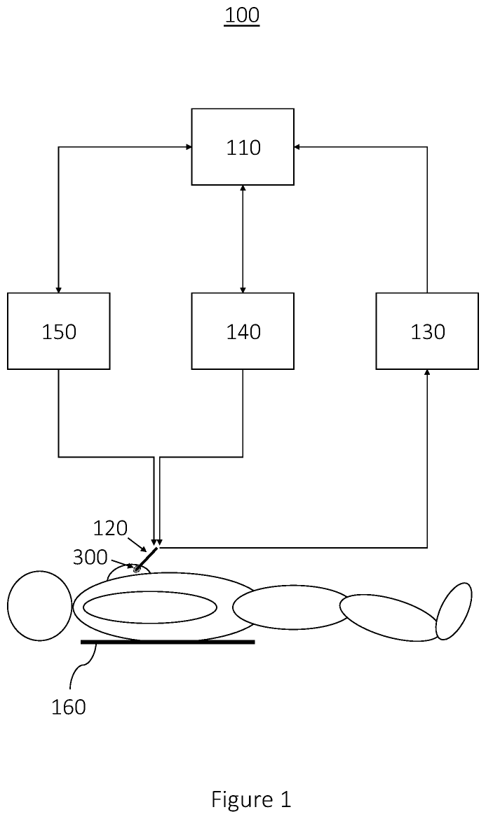

FIG. 1 schematically illustrates a system 100 for curative treatment of a treatment volume 300 comprising at least a part of a cancer tumor, using RFA. The system 100 comprises an electrode arranged on an uninsulated ablating portion 124 of an RF probe 120, and another electrode in the form of an indifferent electrode 160, on which a patient to be treated lies. The system 100 further comprises an RF generating arrangement 150, arranged to supply RF energy to the RF probe 120, so that a current path is established between the RF probe 120 and the indifferent electrode 160 when the RF probe 120 is inserted into a treatment volume 300 inside the patient. This causes the tissue in the treatment volume 300 surrounding the uninsulated ablating portion 124 of the RF probe 120 to be heated, due to the electric resistance of the tissue. If there is good contact between the patient and the indifferent electrode 160, the current density at the indifferent electrode 160 will be much lower than at the RF probe 120, and thus there will be virtually no heating of the skin that is in contact with the indifferent electrode 160. The treatment volume 300 preferably comprises at least a part of a tumor, e.g. a breast tumor. It should preferably be ensured that the RF probe 120, once inserted into the tumor, is not moved in relation to the tumor. In order to ensure this, it is possible to use either an arrangement that keeps the patient still, or an arrangement that ensures that the RF probe 120 moves with the patient, if the patient moves.

The system 100 illustrated in FIG. 1 further comprises a control arrangement 110, a temperature measuring arrangement 130, and a cooling arrangement 140, which is arranged to cool the RF probe 120. The temperature measuring arrangement 130 is arranged to measure an RF probe temperature and based on this provide estimations of the treatment volume temperature T to the control arrangement 110, so that the control arrangement 110 can monitor that the treatment volume temperature T is maintained in the thermotherapy temperature range (60-90° C.) within the whole treatment volume 300. Without cooling of the RF probe 120, it may not be possible to maintain a high enough temperature within the whole treatment volume 300 without the temperature at the surface of the RF probe 120 becoming so high that there is a risk that e.g. coagulated tissue explodes. Thanks to the cooling of the RF probe 120, the maximum temperature within the treatment volume will not be adjacent to the RF probe 120, but further out in the treatment volume 300. The control arrangement 110 controls the cooling arrangement 140 to cool the RF probe 120.

The control arrangement 110 also controls the output power from the RF generating arrangement 150 to the RF probe 120. The output power of the RF generating arrangement 150 regulates the temperature that is generated within the treatment volume 300. The control arrangement 110 controls the RF generating arrangement 150 to supply the amount of RF energy to the RF probe 120 that is needed to maintain a desired treatment volume temperature T_{D}, based on the temperature T estimated by the temperature measuring arrangement 130. The control is preferably PID based.

FIG. 2b schematically illustrates an RF probe 120 inserted into a tumor 210. A major advantage to using the thermotherapy temperature range of 60-90° C. is that the temperature will be raised also in an adjoining volume 200 outside of the treatment volume 300. If the temperature within the whole treatment volume 300 is at least 60° C., the temperature within an adjoining volume 200 will be at least e.g. 50° C., and thus it is ensured that tumor cells within the adjoining volume 200 are also killed during the RFA treatment. Since tumor cells are more sensitive to heat than non-tumor cells, most non-tumor cells in the adjoining volume 200 that are damaged will be able to repair themselves. This is a major advantage to RFA treatment as compared to surgery—a surgeon always has to make the choice to either remove too much tissue to ensure that no tumor cells remain, or risk not removing all tumor cells. With RFA, there is a “safety margin” because of the raised temperature in the adjoining volume 200.

The RF generating arrangement 150 may comprise any number and type of devices that enable a controlled amount of RF energy to be supplied to the RF probe 120. In embodiments, the RF generating arrangement 150 comprises an oscillator that supplies a frequency of e.g. 1 MHZ, an RF amplifier that may supply e.g. up to 300 W, and a balanced modulator that controls the output from the RF amplifier between 0 and 100%. In this case, the control arrangement 110 may control the balanced modulator, e.g. using PID control.

One way of monitoring how the tumor treatment is proceeding is to monitor the output power of the RF generating arrangement 150. The blood vessels within the tumor constantly actively transport heat away from the tumor, and thus a relatively high output power (typically around 60-80 W) from the RF generating arrangement 150 will be needed during the first part of the treatment. However, as the heat begins coagulating the blood vessels within the tumor, the output power from the RF generating arrangement 150 that is needed to maintain a desired treatment volume temperature T_{D} becomes lower (typically around 20-30 W). When all the blood vessels within the treatment volume 300 have coagulated, and the tumor cells have thus been denaturized, a lower power is needed. The output power from the RF generating arrangement 150 that is needed to maintain the desired treatment volume temperature T_{D} thus indicates the level of blood vessel coagulation, and thereby to what extent the cells within the treatment volume 300 have been denaturized. The denaturized cells may be left to be later resorbed by the body.

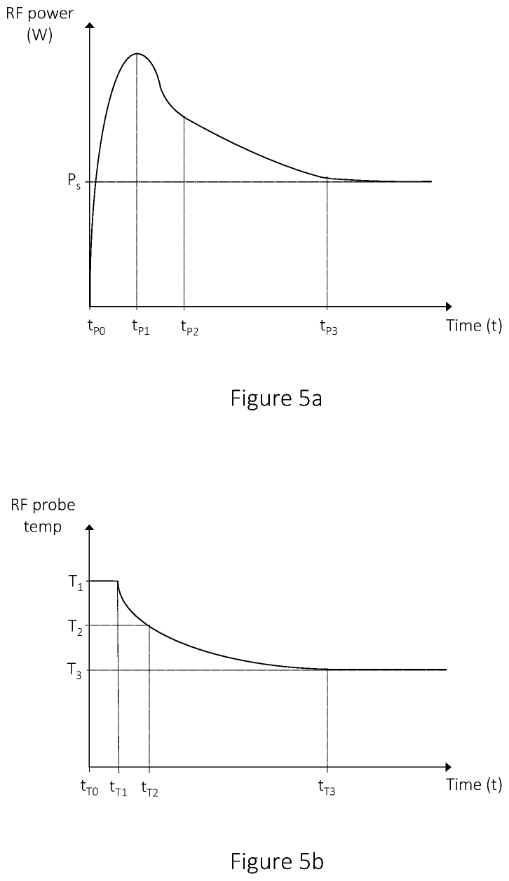

FIG. 5a schematically illustrates how the output power of an RF generating arrangement 150 may vary during the course of an RFA treatment. During the first time period (from t_{P0} to t_{P1}), the output power from the RF generating arrangement 150 typically rises sharply, in order for the treatment volume to be heated to a treatment volume temperature T_{D} desired for the RFA treatment (typically 60-90° C.). When the treatment volume has reached the desired treatment volume temperature T_{D}, the output power from the RF generating arrangement 150 is gradually lowered to an output power needed to maintain the desired treatment volume temperature T_{D}. As can be seen in FIG. 5a, after a certain time (t_{P2}), the output power from the RF generating arrangement 150 is typically lowered more or less linearly. This indicates the gradual coagulation of the blood vessels within the tumor. At a final point in time (t_{P3}), the output power from the RF generating arrangement 150 is no longer lowered, but instead more or less stabilized at a certain level P_{S}. This indicates that all the blood vessels within the tumor have coagulated, and no longer actively transport heat away from the tumor. The output power from the RF generating arrangement 150 that is needed to maintain the desired treatment volume temperature T_{D} is then typically more or less constant.

At the point in time when the output power from the RF generating arrangement 150 that is needed to maintain the desired treatment volume temperature T_{D} becomes more or less constant, the tumor is denaturized, and the curative treatment is thus finished. By determining the point in time (t_{P3}) when the curve stabilizes, it can be determined when the RF generating arrangement 150 can be turned off, thus ending the RFA treatment, based on blood vessel coagulation control. One way of determining this point in time (t_{P3}) is to analyze the curve and determine when there is no longer any substantial lowering of the output power from the RF generating arrangement 150. In embodiments, the determining of the point in time when there is no longer any substantial lowering of the output power from the RF generating arrangement 150 involves determining when the derivative of the output power from the RF generating arrangement 150 comes within a predetermined threshold, close to zero. At this point in time, t_{P3}, the blood vessels within the treatment volume 300 have coagulated, and the tumor has therefore been destroyed in its entirety. The RF generating arrangement 150 can then be turned off, thus ending the RFA treatment.

Since the derivative of the output power from the RF generating arrangement 150 will be zero also early during the treatment process, at the high point of the curve at the point in time t_{P1}, any automatic system for determining when there is no longer any substantial lowering of the output power from the RF generating arrangement 150 based on the derivative of the output RF power curve needs to differentiate between these points. This may be done e.g. based on a certain treatment time having passed, or based on the derivative first being negative.

The above described concept for determining when the treatment may be ended is entirely independent of how the treatment volume temperature T is estimated.

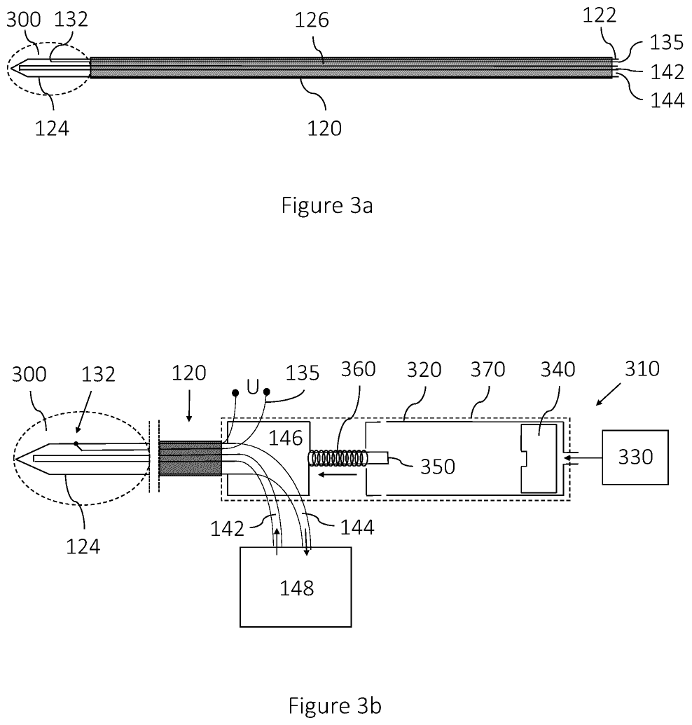

FIGS. 3a-b illustrate aspects of one or more embodiments of an RF probe 120 adapted to be inserted into a tumor and heat tissue within a e.g. spheroid treatment volume 300 around the uninsulated ablating portion 124 of the RF probe 120. The RF probe 120 may e.g. comprise a hollow metal tube, e.g. made of stainless steel, which may be covered along its length by an insulating covering 126, made of an insulating material such as e.g. a rubber or plastic material. The insulating covering 126 ensures that only the ablating portion 124 of the RF probe is in contact with the surrounding tissue. The ablating portion 124 of the RF probe 120 is adapted to be inserted into the tumor, and is therefore not covered by any insulating covering 126.

The size of the treatment volume 300 depends on the length of the uninsulated ablating portion 124 of the RF probe 120 and the diameter of the RF probe 120. A spheroid treatment volume 300 will have a rotational diameter determined by the diameter of the RF probe 120 and a length determined by the length of the uninsulated ablating portion 124 of the RF probe 120. The length of the uninsulated ablating portion 124 and the diameter of the RF probe 120 are therefore preferably adapted to the size of the tumor to be treated. The RF probe diameter that corresponds to a particular treatment volume rotational diameter, and the ablating portion length that corresponds to a particular treatment volume length, may e.g. be determined empirically. The length of the uninsulated ablating portion 124 may e.g. be 10-30 mm, and the diameter of the RF probe 120 may e.g. be 1-3 mm, e.g. around 1.5 mm.

If the insulating covering 126 is movable along the length of the RF probe 120, the length of the uninsulated ablating portion 124 can be adapted. In this way, the same RF probe 120 may be used for tumors of different sizes and/or shapes (although for some tumor sizes and/or shapes a different RF probe 120 having a different diameter may be needed). The movement of the insulating covering 126 may e.g. be effected by simply pushing the insulating covering 126 manually along the length of the RF probe 120. However, there is in such an embodiment preferably enough friction between the hollow metal tube and the insulating covering 126 to ensure that the insulating covering 126 is not unintentionally moved when the RF probe 120 is inserted into the tumor.

The system 100 preferably comprises a temperature measuring arrangement 130, arranged to measure the RF probe temperature, and based on this estimate the temperature T within the treatment volume 300. The estimation of the treatment volume temperature T based on the measured RF probe temperature may be made by processing means comprised in the temperature measuring arrangement 130, or by processing means comprised in the control arrangement 110. The RF probe temperature is preferably measured by a temperature sensing arrangement comprised in the temperature measuring arrangement 130. The temperature sensing arrangement may e.g. comprise a temperature sensor 132, arranged at the uninsulated ablating portion 124 of the RF probe 120.

In one embodiment, the temperature sensing arrangement is arranged to measure the RF probe temperature using a thermocouple. A thermocouple is an electrical device consisting of two dissimilar electrical conductors forming an electrical junction. A thermocouple produces a temperature-dependent voltage that can be converted into a temperature measurement. If the hollow metal tube is made of stainless steel, and a conductor 135 made of the metal alloy constantan is arranged inside the RF probe 120 and connected to the hollow metal tube at a point 132 on the uninsulated ablating portion 124 of the RF probe 120, the voltage U between the hollow metal tube and the conductor 135 can be converted into the RF probe temperature based on the factor 0.050 mV/° C. (which applies to a thermocouple consisting of iron and constantan). The conductor 135 however needs to be insulated from the hollow metal tube from the connection point 132 to the point where the voltage is measured. The conductor may e.g. by glued to the inside of the hollow metal tube using a resin that insulates it from the hollow metal tube. This also makes the arrangement of the conductor 135 mechanically stable.

The RF probe 120 preferably also comprises a cooling liquid supply channel 142, arranged inside the hollow metal tube to supply cooling liquid to the uninsulated ablating portion 124 of the RF probe 120. The cooling liquid may e.g. be supplied to the cooling liquid supply channel 142 at the connecting end 122 of the RF probe 120. As illustrated in FIG. 3b, the connecting end 122 of the RF probe 120 may comprise a distribution unit 146, in which all the connection points to external systems are arranged. The distribution unit 146 may e.g. be arranged to have a shape that makes it easy to hold during insertion of the RF probe 120 into the patient, so that it becomes easy for the physician to hold the RF probe 120 and apply manual force to it in order to insert it into the tumor.

The cooling liquid supply channel 142 preferably runs almost all the way to the tip of the RF probe 120, inside the hollow metal tube. This ensures that the cooling liquid is supplied to the whole of the uninsulated ablating portion 124 of the RF probe 120. The cooling liquid may then flow freely back inside the hollow metal tube, to be collected in a cooling liquid return channel 144 connected to the RF probe 120. If there is a conductor 135 arranged inside the hollow metal tube, this conductor 135 is preferably separated from the cooling liquid, e.g. by being covered by the resin that is used to glue it to the inside of the hollow metal tube.

The cooling liquid is preferably cooled, e.g. using a Peltier cooler, before it is returned to the cooling liquid supply channel 142. The temperature to which the cooling liquid is cooled may be constant, or adapted to the cooling need. The cooling liquid may e.g. be water. The cooling liquid is preferably circulated through the cooling liquid supply channel 142, the hollow tube, the cooling liquid return channel 144 and the cooler, e.g. by being pumped with a pump, e.g. a peristaltic pump (which has the advantage that no contaminations are gathered in the pump). FIG. 3b schematically illustrates an arrangement 148 for pumping and cooling the cooling liquid.

It is important that the RF probe 120 is inserted into the tumor without dislocating the tumor. The treatment of a tumor is often based on first determining the location of the tumor, e.g. using medical imaging technology such as e.g. CT, MRI, and/or ultrasound. If the RF probe 120 dislocates the tumor during insertion into the tumor, the location of the tumor will no longer correspond to the determined location.

If the RF probe 120 is just pushed into the tumor, there is a high risk that the tumor will be pushed away from the RF probe 120 instead of being penetrated by the RF probe 120, since a tumor is often denser and/or harder than the surrounding tissue. For this reason, the system 100 preferably comprises a striking arrangement 310 that is arranged to push the RF probe 120 into the tumor in distinct strokes when the physician applies manual force to the RF probe 120 in order to insert it into the tumor. Each stroke by the striking arrangement 310 pushes the RF probe 120 forward a short distance with a very high acceleration, and there is an interval between each stroke.

In the embodiment illustrated in FIG. 3b, the striking arrangement 310 comprises an impact housing 320, a compressed air source 330 and a striking device 340. The compressed air source 330 is preferably arranged to release compressed air pulses that “shoot” the striking device 340 towards an impact peg 350 that extends from the connecting end 122 of the RF probe 120 into an opening in the impact housing 320. The elastic impact between the striking device 340 and the impact peg 350 pushes the RF probe 120 forward a short distance with a very high acceleration. In this arrangement, the connecting end 122 of the RF probe 120 can only be pushed a distance defined by the length that the impact peg 350 extends into the impact housing 320.

Since the striking arrangement 310 is filled with air, this air will act as an “air cushion” for the striking device 340, so that the striking device 340 will not hit the end of the striking arrangement 310 too hard (which could cause vibrations). The impact housing 320 is preferably arranged to allow the air to escape from the impact housing 320 after each compressed air pulse. There is preferably also some means, such as e.g. an air valve for compressed air, arranged for ensuring that the striking device 340 is returned to the starting position after each strike. There may also be some means, such as e.g. a magnet, arranged for ensuring that the striking device 340 stays in the starting position when it is not shot towards the impact peg 350.

The striking arrangement 310 preferably comprises a spring device 360, arranged to interact with the impact peg 350. The spring device 360 pushes the RF probe 120 away from the striking arrangement 310. When no force is applied to the RF probe 120, the spring device 360 will not be very compressed, and the impact peg 350 will thus not extend far enough into the impact housing 320 to engage with the striking device 340. Thus, the RF probe 120 will not be pushed forward by the striking device 340 when no force is applied to the RF probe 120.

However, when the RF probe 120 is pushed towards tissue in a patient, the spring device 360 is compressed to an extent that is proportional to the applied force. Thus, the length that the impact peg 350 extends into the impact housing 320 will depend on the force applied to the RF probe 120 to counteract the spring force in the spring device 360. This means that the amplitude of the striking of the striking arrangement 310 will be proportional to the force applied to the RF probe 120. Such an arrangement ensures that the RF probe 120 does not move when no force is applied.

It is advantageous that the amplitude of the striking of the striking arrangement 310 is proportional to the force applied to the RF probe 120, because this is an easy way for the physician to use tactile feedback to control the insertion of the RF probe 120 into the tumor. This increases the tactility of the insertion. If the patient e.g. feels pain, the physician can simply stop applying force to the RF probe 120, and the RF probe 120 will then no longer be pushed into the tumor. In other embodiments of the striking arrangement 310, there may be other means for ensuring that the amplitude of the striking of the striking arrangement 310 is proportional to the force applied to the RF probe 120.

The compressed air source 330 is preferably arranged to release a new compressed air pulse after a predetermined time, to once again shoot the striking device 340 towards the impact peg 350. Thereby, the RF probe 120 is pushed into the tumor in distinct strokes. The frequency at which the compressed air source 330 releases the compressed air pulses may also be adapted to the force applied to the RF probe 120 to push it into the tumor. This force may be measured using any suitable sensor, e.g. a sensor that senses the compression of the spring device 360.

In the embodiment illustrated in FIG. 3b, the distribution unit 146 and the impact housing 320 are held together by a schematically illustrated rig arrangement 370, that keeps them aligned. In such an embodiment, the whole rig arrangement 370 may be held by the physician during insertion of the RF probe 120 in a patient. However, an alternative arrangement for transferring an impact from a striking arrangement 310 to an RF probe 120 is illustrated in the article “A new method to gently place biopsy needles or treatment electrodes into tissues with high target precision”, Phys Med 2016 May, 32(5):724-7, by Wiksell, H. et al. In such an embodiment, the RF probe 120 may be held by the physician and the striking arrangement 310 may be held by support personnel, and they need not be aligned.

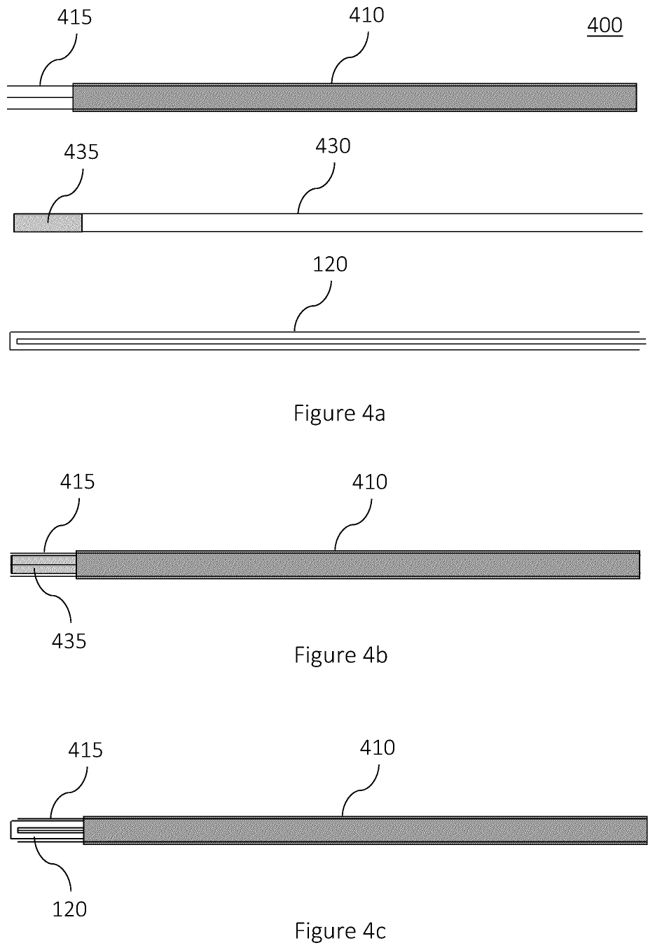

In embodiments, the above described RF probe 120 may also be used in an arrangement for tissue sampling. FIGS. 4a-c illustrate a probe arrangement 400 for use in curative treatment of cancer tumors. The probe arrangement 400 preferably comprises an insertion tube 410, an open ended biopsy needle 430 and an RF probe 120.

When the probe arrangement 400 is to be used for tissue sampling, the biopsy needle 430 is inserted into the insertion tube 410, as illustrated in FIG. 4b. The probe arrangement 400 is then pushed into the tumor, in the same way as described above for the RF probe 120. The probe arrangement 400 preferably comprises a striking arrangement that is arranged to push the probe arrangement 400 into the tumor in distinct strokes with high acceleration (e.g. the above described striking arrangement 310). As the probe arrangement 400 is pushed into the tumor, a tissue sample 435 from the tumor is pushed into the biopsy needle 430. Thanks to the distinct pushing of the probe arrangement 400 into the tumor, the tissue sample 435 will be preserved in the biopsy needle 430.

If the acceleration is high enough (e.g. 100,000 g) during the insertion of the biopsy needle 430 into the tumor, the acceleration/inertia forces that the cutting edge of the biopsy needle 430 submit to the tissue that is being cut through may in itself kill tumor cells and thus prevent viable tumor seeding caused by the tissue sampling. However, anti-seeding may also be provided using RF energy. In embodiments, the biopsy needle 430 is connected to the RF generating arrangement 150. If the biopsy needle 430 is insulated along its whole length except for at the cutting edge, a very localized current can be generated around the cutting edge of the biopsy needle 430. This current generates localized heat that will kill tumor cells close to the cutting edge, and thus prevent tumor seeding. In order to ensure that the tissue block inside the biopsy needle is not affected, the RF energy is preferably provided in very short pulses. The biopsy needle 430 will act as a Faraday cage and protect the tissue block inside.

When the biopsy needle 430 has been retracted from the insertion tube 410, the RF probe 120 may be inserted instead. Since the biopsy needle 430 has taken a cylindrical tissue sample and thus left a cavity in the tumor, it is not necessary for the RF probe 120 in this embodiment to have a pointed end (but a pointy RF probe 120 may of course be used anyhow). The biopsy needle 430 may have a diameter that is slightly smaller than the RF probe 120, to ensure that there is contact between the RF probe 120 and the surrounding tissue.

The probe arrangement 400 may comprise means to create a negative pressure in the cavity when the RF probe 120 has been inserted, since this creates better contact between the RF probe 120 and the surrounding tissue. There may e.g. be a channel, that is connected to a vacuum pump, arranged in the insertion tube 410, so that the surrounding tissue may be sucked towards the RF probe 120. If the vacuum pump is active also during the tissue sampling, any tumor cells that may be loosened when the biopsy needle 430 cuts through the tumor will be sucked into the insertion tube 410, thus lowering even further the risk of tumor seeding.

The RF probe 120 may comprise all the features described in connection with FIGS. 3a-b, even though this is not illustrated here.

There are many advantages to using the same probe arrangement 400 for both tissue sampling and RFA treatment:
Since the insertion tube 410 remains in its inserted position until the RFA treatment is finished, it is ascertained that the tissue sample is taken from the position in the tumor that is treated.
Since the RFA treatment will kill the cancer cells around the uninsulated ablating portion 124 of the RF probe 120, and the insertion tube 410 will not be retracted from the tumor until after the RFA treatment, the risk of the biopsy causing tumor seeding is lowered considerably.
The biopsy needle 430 may also be connected to the RF generating arrangement 150, and thus generate localized heat close to the cutting edge, which will further lower the risk of the tissue sampling causing tumor seeding.
Since the biopsy needle 430 leaves a cavity in the tumor, it is not necessary for the RF probe 120 to have a pointed end.

As explained above, there are also distinct advantages to inserting the biopsy needle 430 together with the insertion tube 410, as compared to if the biopsy needle 430 would be inserted into the insertion tube 410 when insertion tube 410 has already been inserted into in the tumor. Thanks to the distinct pushing of the probe arrangement 400 into the tumor, the tissue sample 435 will be preserved in the biopsy needle 430. Further, if the acceleration is high enough during the insertion of the biopsy needle 430 into the tumor, the acceleration Inertia forces that the cutting edge of the biopsy needle 430 submit to the tissue that is being cut through may in itself kill tumor cells, and thus prevent viable tumor seeding caused by the tissue sampling. This would not be the case if the biopsy needle 430 would not be inserted into the insertion tube 410 when this is pushed into the tumor.

The distal portion of the insertion tube 410 may comprise a directing arrangement 415, arranged to correctly direct the RF probe 120 into the cavity created by the biopsy needle 430. Such a directing arrangement 415 may e.g. comprise thin bars extending out from the insertion tube 410. Such bars may also help keeping the cavity open between the retraction of the biopsy needle 430 and the insertion of the RF probe 120. However, the insertion tube 410 may also simply be shorter than the biopsy needle 430 and the RF probe 120, and not comprise any such directing arrangement 415.

The above described probe arrangement 400 may be used in any system 100 for RFA treatment, entirely independent of e.g. how the treatment volume temperature T is estimated.

It is important to control the temperature in the tumor during an RFA treatment. However, the temperature will not be the same within the whole treatment volume 300. The current density is highest at the surface of the RF probe 120, and falls further out in the treatment volume 300. Since the heating is caused by the electric resistance of the tissue, the generated heat is proportional to the current density. Without cooling of the RF probe 120, it may not be possible to maintain a high enough temperature within the whole treatment volume 300 without the temperature at the surface of the RF probe 120 becoming so high that there is a risk that e.g. coagulated tissue explodes. Thanks to the cooling of the RF probe 120, the maximum temperature within the treatment volume will not be adjacent to the RF probe 120, but further out in the treatment volume 300.

It is difficult to accurately measure the temperature within the treatment volume 300, and it is therefore preferred to estimate the treatment volume temperature T based on a measured RF probe temperature. However, in order to estimate an accurate treatment volume temperature T, it is desirable to allow the temperature within the treatment volume 300 to equalize into a steady state before measuring the RF probe temperature. If both the RF generating arrangement 150 and the cooling arrangement 140 are shut off, the temperature within the treatment volume 300 will no longer be affected by any external energy supply, and will thus be allowed to equalize. A temperature measured at the surface of the RF probe 120 will then roughly correspond to the temperature within the whole treatment volume 300.

One way of measuring a temperature at the surface of the uninsulated ablating portion 124 of the RF probe 120 that roughly corresponds to the treatment volume temperature T is therefore to temporarily shut off both the RF generating arrangement 150 and the cooling arrangement 140 for a selected “shut-off period”. When there is no current heating the treatment volume 300 and no cooling arrangement 140 cooling the probe and adjacent parts of the treatment volume 300, the treatment volume temperature T will equalize, and thus roughly correspond to the temperature at the surface of the uninsulated ablating portion 124 of the RF probe 120, i.e. the RF probe temperature. Since these shut-offs make the total treatment time longer, the shut-off periods should preferably be as short as possible.

FIG. 5b schematically illustrates how an RF probe temperature (measured at the uninsulated ablating portion 124 of the RF probe 120) is typically affected by the RF generating arrangement 150 and the cooling arrangement 140 being turned off. When the RF generating arrangement 150 heats the treatment volume 300 and the cooling arrangement 140 cools the RF probe 120, the measured RF probe temperature will typically be different from the general treatment volume temperature T, e.g. T₁, as illustrated in FIG. 5b. The measured RF probe temperature may be either higher or lower than the treatment volume temperature T, depending on the location of the temperature sensing arrangement and the amount of cooling applied to the RF probe 120.

If the RF generating arrangement 150 and the cooling arrangement 140 are turned off at a time t_{T1}, the RF probe temperature will typically change slowly. After a long enough time (t_{T3}), the RF probe temperature will typically stabilize at a temperature T₃ (which roughly corresponds to the treatment volume temperature T). However, since the time dependence of thermal systems can be approximated to follow dynamics of the first order, it is possible to estimate the final temperature T₃ based on the shape of the measured RF probe temperature curve, e.g. by determining the derivative of the measured RF probe temperature curve up to a time t_{T2}. This means that it is not necessary to wait for the RF probe temperature to fully stabilize, and thus the shut-off period can be substantially shortened.

In embodiments, the RF generating arrangement 150 and the cooling arrangement 140 are simultaneously turned off for a selected shut-off period at selected intervals. Thereby, it is possible for the temperature measuring arrangement 130 to regularly estimate the equalized treatment volume temperature T. The selected shut-off period and intervals may e.g. be adapted for the specific patient based on previous treatments, or they may be adapted during the course of the treatment, e.g. based on the amount of variation in the measured RF probe temperature. A typical shut-off period may e.g. be around 20 seconds. The intervals are preferably selected so as to allow a close control of the treatment, without unnecessarily affecting the efficiency of the treatment. Typical intervals may e.g. be 1-2 minutes. The total treatment time may e.g. be 5-20 minutes.

This enables a precise control of the treatment volume temperature T.

The RF probe temperature is preferably measured by a temperature sensing arrangement comprised in the temperature measuring arrangement 130. The estimation of the treatment volume temperature T based on the measured RF probe temperature may be made by processing means comprised in the temperature measuring arrangement 130, or by processing means comprised in the control arrangement 110.

The turning off of the cooling arrangement 140 may involve just turning off the circulation of the cooling liquid, so that the cooling liquid is no longer cooled.

The system 100 may also comprise a skin temperature sensor, e.g. an infrared thermometer, for measuring the temperature of the skin of the patient, close to the RF probe 120. In some situations, especially when the tumor is located near the surface of the body, the RF energy may heat also the skin. It may then be necessary to cool the skin, to prevent skin damage due to the heat. Any damage to the skin increases the risk of infections, and infections lower the chances of the treatment being successful. The output from the skin temperature sensor may be provided to the control arrangement 110, which may automatically control the cooling of the skin.

The control arrangement 110 preferably comprises a display, on which information showing the various parameters that are monitored and regulated by the control arrangement 110 are displayed, e.g. in the form of curves in different colors. An operator may monitor these curves, and adapt the treatment based on them. The operator may e.g. lower the power supplied by the RF generating arrangement 150 if the skin of the patient starts becoming uncomfortably hot, or raise the power if it appears that the patient can withstand this. The control arrangement 110 may alternatively be entirely automatic, and control the various parameters based on preprogrammed parameters. The control arrangement 110 may e.g. be a computer.

In order for the system to fulfil regulations such as IEC 60-601-1, there is a need for ascertaining that the patient leakage current (PLC) in no situation can exceed the value set according to the regulations (e.g. 10 μA). One way of ascertaining this is to arrange an isolation transformer between the RF generating arrangement 150 and the RF probe 120. Use of an isolation transformer ensures that there is no conductive path between the RF generating arrangement 150 and the RF probe 120.

### Method Embodiments

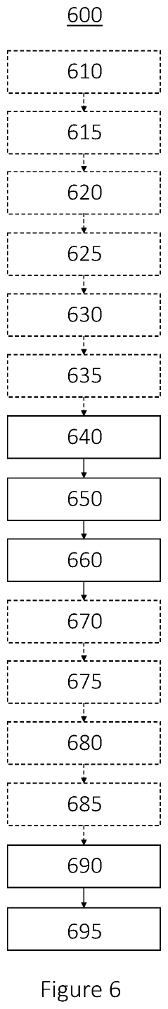

FIG. 6 schematically illustrates a method 600, in a system 100 for curative RFA treatment of a treatment volume 300 comprising at least a part of a cancer tumor by applying RF energy between an electrode arranged on an uninsulated ablating portion 124 of an RF probe 120 and another electrode (e.g. an indifferent electrode 160), for determining the point in time t_{P3} when the blood vessels within the treatment volume 300 have coagulated. The method 600 may comprise:
Step 640: measuring an RF probe temperature using a temperature measuring arrangement 130, and based on this estimating the treatment volume temperature T.
Step 650: cooling the RF probe 120, using a cooling arrangement 140.
Step 660: supplying, based on the estimated treatment volume temperature T, the amount of RF energy to the RF probe 120 that is needed for a desired treatment volume temperature T_{D} to be maintained, using an RF generating arrangement 150.
Step 690: monitoring the RF energy supplied by the RF generating arrangement 150 to the RF probe 120.
Step 695: determining the point in time t_{P3} when there is no longer any substantial lowering of the output power from the RF generating arrangement 150, in order to end the RFA treatment by turning off the RF generating arrangement 150 after this point in time t_{P3}.

This is a precise way of determining that the tumor has been denaturized, and that the curative treatment is thus finished.

This point in time t_{P3} may e.g. be determined by determining when the derivative of the output power from the RF generating arrangement 150 comes within a predetermined threshold, close to zero. Since the derivative of the output power from the RF generating arrangement 150 will be zero also early during the treatment process, at the high point of the curve at the point in time t_{P1}, any automatic system for determining when there is no longer any substantial lowering of the output power from the RF generating arrangement 150 based on the derivative of the output RF power curve needs to differentiate between these points. This may be done e.g. based on a certain treatment time having passed, or based on the derivative first being negative.

The estimation of the treatment volume temperature T may be made in the temperature measuring arrangement 130, or in the control arrangement 110, based on measurements provided by the temperature measuring arrangement 130.

In embodiments, the method 600 further comprises at least one of the following:
Step 610: arranging the temperature measuring arrangement 130 to comprise a thermocouple comprising a conductor 135 that is arranged inside the RF probe 120 and connected to the uninsulated ablating portion 124 of the RF probe 120. This is a simple temperature measuring arrangement.
Step 615: arranging a cooling liquid supply channel 142 inside the RF probe 120, to supply cooling liquid to the uninsulated ablating portion 124 of the RF probe 120. This is a simple way of controlling the cooling of the RF probe.
Step 620: arranging a striking arrangement 310 to push the RF probe 120 into the tumor in distinct strokes. The striking arrangement 310 may be arranged to comprise a striking device 340, arranged within an impact housing 320 to be shot towards an impact peg 350 that extends from a connecting end 122 of the RF probe 120, so that the impact between the striking device 340 and the impact peg 350 pushes the RF probe 120 forward, with a very high acceleration, a distance that is defined by the length that the impact peg 350 extends into the impact housing 320. The striking arrangement 310 may further be arranged to comprise a spring device 360, with which the impact peg 350 interacts, wherein the spring device 360 pushes the RF probe 120 away from the striking arrangement 310, so that the length that the impact peg 350 extends into the impact housing 320 becomes dependent of the force applied to the RF probe 120 to counteract the spring force in the spring device 360. This is a simple yet efficient way of arranging a striking arrangement.
Step 625: For an RF probe 120 arranged to be comprised in a probe arrangement 400 also comprising an insertion tube 410 and a biopsy needle 430, arranging the insertion tube 410 to be inserted into a tumor together with the biopsy needle 430.
Step 630: For an RF probe 120 arranged to be comprised in a probe arrangement 400 also comprising an insertion tube 410 and a biopsy needle 430, allowing the insertion tube 410 to remain inserted in the tumor when the biopsy needle 430 is retracted.
Step 635: For an RF probe 120 arranged to be comprised in a probe arrangement 400 also comprising an insertion tube 410 and a biopsy needle 430, allowing the insertion of the RF probe 120 into the cavity in the tumor created by the biopsy needle 430.
This enables the taking of a tissue sample in the exact location that is treated with the curative RFA treatment.
Step 670: shutting off the RF generating arrangement 150 and the cooling arrangement 140 for a selected shut-off period, to thereby allow the treatment volume temperature T to equalize, so that it can be more accurately estimated by the temperature measuring arrangement 130. This enables a precise control of the treatment temperature.
Step 675: estimating the treatment volume temperature T based on the shape of the measured RF probe temperature curve, e.g. based on a determination of the derivative of the measured RF probe temperature curve up to the end of the shut-off period. This means that it is not necessary to wait for the RF probe temperature to fully stabilize, and thus the shut-off period can be substantially shortened.
Step 680: circulating cooling liquid to cool the RF probe 120.
Step 685: turning off the cooling arrangement 140 by stopping the circulation of the cooling liquid. This is a simple way of controlling the cooling of the RF probe 120.

### Use Case

A typical use case is a patient that has been diagnosed with breast cancer that has not yet spread outside of a local primary tumor in a breast. In order to curatively treat the tumor using RFA, it is first necessary to determine the exact size, shape and location of the tumor, generally using some kind of medical imaging technology, such as e.g. CT, MRI, and/or ultrasound. In order to treat the tumor using RFA, a e.g. spheroid treatment volume 300 having a certain rotational diameter and a certain length then needs to be defined. The treatment volume 300 preferably encompasses the whole tumor, with some margin.

An RF probe 120 that has a diameter that causes the treatment volume 300 to have the desired rotational diameter then needs to be selected from a set of preferably single-use sterile RF probes 120 with different diameters. The length of the uninsulated ablating portion 124 of the selected RF probe 120 then needs to be adjusted to cause the treatment volume 300 to have the desired length. If the length of the uninsulated ablating portion 124 of the RF probes 120 is not adjustable, the set of RF probes 120 needs to comprise RF probes 120 having different combinations of diameters and ablation lengths.

If the tumor has a shape that cannot easily be encompassed into a spheroid treatment volume 300, it is possible to use a number of different RF probes 120 simultaneously and thereby create a treatment volume 300 of any desired shape.

The RF probe 120 is inserted into the tumor, preferably using some kind of striking arrangement 310, to ensure that the RF probe 120 is pushed into the tumor in distinct strokes with high acceleration. If the amplitude of the striking of the striking arrangement 310 is proportional to the manual force applied to the RF probe 120 by the physician, this gives the physician tactile feedback during the insertion of the RF probe 120 into the tumor. This increases the tactility of the insertion. If the RF probe 120 and the striking arrangement 310 are held together by a rig arrangement 370 that keeps them aligned, the whole rig arrangement 370 may be held by the physician. However, in other embodiments, the RF probe 120 may be held by the physician and the striking arrangement 310 may be held by support personnel. However, it is in such situations important that it is still the physician holding the RF probe 120 that controls the insertion.

In embodiments where the RF probe 120 is comprised in a probe arrangement 400 also comprising an insertion tube 410 and a biopsy needle 430, the treatment session may begin with the taking of a tissue sample. In this case, the biopsy needle 430 is first arranged in the insertion tube 410, and the probe arrangement 400 is pushed into the tumor in distinct strokes with high acceleration, as explained above. When the biopsy needle 430 is retracted, the insertion tube 410 remains inserted, to allow the RF probe 120 to be inserted into the cavity in the tumor created by the biopsy needle 430.

When the RF probe 120 has been inserted into the predetermined treatment position, the RFA treatment is started by the RF generating arrangement 150 being turned on. The estimated temperature within the treatment volume 300 is then monitored throughout the treatment, and the output power from the RF generating arrangement 150 is continuously adapted so that the temperature within the treatment volume 300 is kept within the desired temperature range (60-90° C.). When the output power from the RF generating arrangement 150 stabilizes, this indicates that the treatment is finished.

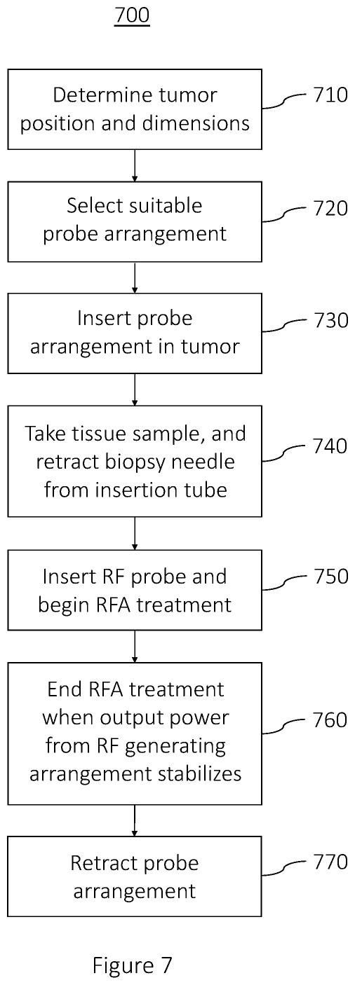

A typical use case that includes the taking of a tissue sample may thus be described as a method 700 for curative treatment of a treatment volume 300 comprising at least a part of a cancer tumor by applying RF energy between an electrode arranged on an RF probe 120 and another electrode, e.g. an indifferent electrode 160. The method 700, which is schematically illustrated in FIG. 7, comprises the following steps:
Step 710: Determine the tumor position and dimensions (after first diagnosing a tumor suitable for RFA).
Step 720: Select a suitable probe arrangement 400.
Step 730: Insert the probe arrangement 400 into the tumor, with the biopsy needle 430 inserted into the insertion tube 41.
Step 740: Take a tissue sample, and then retract the biopsy needle 430 from the insertion tube 410.
Step 750: Insert the RF probe 120 into the insertion tube 410, and begin the RFA treatment.
Step 760: End the RFA treatment when the output power from the RF generating arrangement 150 has stabilized.
Step 770: Retract the probe arrangement 400 from the patient.

It is often necessary to use local anesthesia before inserting the RF probe 120 into the tumor. It is however important to ensure that the delivering of such local anesthesia does not cause tumor seeding.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. The disclosure refers to a number of different curves. It is to be understood that actual measured curves are not as smooth as the schematic curves described, and thus determinations of e.g. derivatives of curves normally involve first smoothing the curves, e.g. by filtering them to remove the high frequency fluctuations. In the disclosure, a number of different concepts are described. Each of these concepts may be used alone, or in any combination with any of the other concepts. Each inventive concept may thus be claimed as a separate invention. Although the disclosure describes the concepts in relation to monopolar RFA treatments, the concepts may also be used in bipolar RFA treatments. If the RFA treatment is a monopolar RFA treatment, the other electrode is preferably in the form of an indifferent electrode. If the RFA treatment is a monopolar RFA treatment, the other electrode may be arranged on a separate RF probe, or on an uninsulated ablating portion of the same RF probe. The RFA treatment may also be a combination of a monopolar RFA treatment and a bipolar RFA treatment. The RFA treatment may treat tumors in humans as well as animals. Further, not all of the steps of the claims have to be carried out in the listed order. All technically meaningful orders of the steps are covered by the claims. Accordingly, the scope of the invention is defined only by the claims.

1. A system for curative radio frequency ablation (RFA) treatment of a treatment volume comprising at least a part of a cancer tumor by applying radio frequency (RF) energy between an electrode arranged on an uninsulated ablating portion of an RF probe and another electrode, comprising:
a control arrangement;
a temperature measuring arrangement, arranged to measure an RF probe temperature and based on this provide estimations of the treatment volume temperature (T) to the control arrangement;
a cooling arrangement, arranged to be controlled by the control arrangement to cool the RF probe; and
an RF generating arrangement, controlled by the control arrangement to, based on the estimated treatment volume temperature (T), supply the amount of RF energy to the RF probe that is needed for a desired treatment volume temperature (T_{D}) to be maintained;
wherein the control arrangement is arranged to monitor the RF energy supplied by the RF generating arrangement to the RF probe, and turn off the RF generating arrangement at the point in time (t_{P3}) when there is no longer any substantial lowering of the output power from the RF generating arrangement.

2. The system according to claim 1, wherein the determining of the point in time (t_{P3}) when there is no longer any substantial lowering of the output power from the RF generating arrangement involves determining when the derivative of the output power from the RF generating arrangement comes within a predetermined threshold, close to zero.

3. The system according to claim 1, wherein the control arrangement is arranged to, at selected intervals, shut off the RF generating arrangement and the cooling arrangement for a selected shut-off period, to thereby allow the treatment volume temperature (T) to equalize, so that it can be more accurately estimated by the temperature measuring arrangement.

4. The system according to claim 3, wherein the temperature measuring arrangement is arranged to estimate the treatment volume temperature (T) based on the shape of the measured RF probe temperature curve.

5. The system according to claim 3, wherein the cooling arrangement is arranged to cool the RF probe by circulating cooling liquid, and to be turned off by stopping the circulation of the cooling liquid.

6. The system according to claim 1, wherein the temperature measuring arrangement comprises a thermocouple comprising a conductor that is arranged inside the RF probe and connected to the uninsulated ablating portion of the RF probe.

7. The system according to claim 1, wherein the cooling arrangement comprises a cooling liquid supply channel arranged inside the RF probe to supply cooling liquid to the uninsulated ablating portion of the RF probe.

8. The system according to claim 1, further comprising a striking arrangement, arranged to push the RF probe into the tumor in distinct strokes.

9. The system according to claim 8, wherein the striking arrangement comprises a striking device, arranged within an impact housing to be shot towards an impact peg that extends from a connecting end of RF probe, so that the impact between the striking device and the impact peg pushes the RF probe forward, with a very high acceleration, a distance that is defined by the length that the impact peg extends into the impact housing.

10. The system according to claim 9, wherein the striking arrangement comprises a spring device, with which the impact peg interacts, wherein the spring device pushes the RF probe away from the striking arrangement, so that the length that the impact peg extends into the impact housing becomes dependent of the force applied to the RF probe to counteract the spring force in the spring device.

11. The system according to claim 1, wherein the RF probe is comprised in a probe arrangement also comprising an insertion tube and a biopsy needle, and the insertion tube is arranged to be inserted into a tumor together with the biopsy needle, remain inserted in the tumor when the biopsy needle is retracted, and allow the RF probe to be inserted into the cavity in the tumor created by the biopsy needle.

12. The system according to claim 11, arranged to allow the biopsy needle to be connected to the RF generating arrangement, wherein the biopsy needle is insulated along its length so that the RF energy only generates localized heat around the cutting edge of the biopsy needle.

13. A method, in a system for curative radio frequency ablation (RFA) treatment of a treatment volume comprising at least a part of a cancer tumor by applying radio frequency (RF) energy between an electrode arranged on an uninsulated ablating portion of an RF probe and another electrode, for determining the point in time (t_{P3}) when the blood vessels within the treatment volume have coagulated, comprising:
measuring an RF probe temperature using a temperature measuring arrangement, and based on this estimating the treatment volume temperature (T);
cooling the RF probe, using a cooling arrangement;
supplying, based on the estimated treatment volume temperature (T), the amount of RF energy to the RF probe that is needed for a desired treatment volume temperature (T_{D}) to be maintained, using an RF generating arrangement;
monitoring the RF energy supplied by the RF generating arrangement to the RF probe; and
determining the point in time (t_{P3}) when there is no longer any substantial lowering of the output power from the RF generating arrangement, in order to end the RFA treatment by turning off the RF generating arrangement (150) after this point in time (t_{P3}).

14. The method according to claim 13, wherein the determining of the point in time (t_{P3}) when there is no longer any substantial lowering of the output power from the RF generating arrangement involves determining when the derivative of the output power from the RF generating arrangement comes within a predetermined threshold, close to zero.

15. The method according to claim 13, further comprising shutting off the RF generating arrangement and the cooling arrangement for a selected shut-off period, to thereby allow the treatment volume temperature (T) to equalize, so that it can be more accurately estimated by the temperature measuring arrangement.

16. The method according to claim 15, further comprising estimating the treatment volume temperature (T) based on the shape of the measured RF probe temperature curve.

17. The method according to claim 15, further comprising circulating cooling liquid to cool the RF probe, and turning off the cooling arrangement by stopping the circulation of the cooling liquid.

18. The method according to claim 13, further comprising arranging the temperature measuring arrangement to comprise a thermocouple comprising a conductor that is arranged inside the RF probe and connected to the uninsulated ablating portion of the RF probe.

19. The method according to claim 13, further comprising arranging a cooling liquid supply channel inside the RF probe, to supply cooling liquid to the uninsulated ablating portion of the RF probe.

20. The method according to claim 13, further comprising arranging a striking arrangement to push the RF probe into the tumor in distinct strokes.

21. The method according to claim 20, further comprising arranging the striking arrangement to comprise a striking device, arranged within an impact housing to be shot towards an impact peg that extends from a connecting end of the RF probe, so that the impact between the striking device and the impact peg pushes the RF probe forward, with a very high acceleration, a distance that is defined by the length that the impact peg extends into the impact housing.

22. The method according to claim 21, further comprising arranging the striking arrangement to comprise a spring device, with which the impact peg interacts, wherein the spring device pushes the RF probe away from the striking arrangement, so that the length that the impact peg extends into the impact housing becomes dependent of the force applied to the RF probe to counteract the spring force in the spring device.

23. The method according to claim 13, wherein the RF probe is arranged to be comprised in a probe arrangement also comprising an insertion tube and a biopsy needle, further comprising arranging the insertion tube to be inserted into a tumor together with the biopsy needle, allowing the insertion tube to remain inserted in the tumor when the biopsy needle is retracted, and allowing the insertion of the RF probe into the cavity in the tumor created by the biopsy needle.

24. The method according to claim 23, further comprising arranging the biopsy needle to be connected to the RF generating arrangement, wherein the biopsy needle is insulated along its length so that the RF energy only generates localized heat around the cutting edge of the biopsy needle.
